(19) United States
(12) Patent Application Publication
(10) Pub. No.: US 2002/0155072 A1
(43) Pub. Date: Oct. 24, 2002

(54) Use of polyurethanes for improving the water resistance of cosmetic or dermatological formulations

(75) Inventors: Anja Knuppel, Hamburg (DE); Wiebke Grundt, Buchholz (DE); Jens Schulz, Schenefeld (DE)

Correspondence Address: Bruce S. Londa, Norris McLaughlin & Marcus, 30th Floor, 220 East 42nd Street, New York, NY, 10017, US

(73) Assignee: Beiersdorf Aktiengesellschaft

(21) Appl. No.: 10/017,157

(22) Filed: Dec. 14, 2001

(30) Foreign Application Priority Data
Dec. 18, 2000 (DE) 100 63 130.4

(51) Int. Cl. A61K 7/42

(52) U.S. Cl. 424/59

(57) ABSTRACT

Use of film-forming, water-soluble or water-dispersible polyurethanes for improving the water resistance of cosmetic or dermatological formulations.

### DESCRIPTION

### Use of Polyurethanes for Improving the Water Resistance of Cosmetic or Dermatological Formulations

The present invention relates to cosmetic and dermatological formulations, in particular to cosmetic and dermatological light protection formulations.

The harmful effect of the ultraviolet part of solar radiation on the skin is generally known. The rays have various effects on skin as an organ depending on their particular wavelength: UV-C radiation with a wavelength below 290 nm is absorbed by the ozone layer in the earth's atmosphere and therefore is of no physiological significance. By contrast, rays in the range between 290 nm and 320 nm, the UV-B region, cause erythema, simple sunburn or even burns of greater or lesser severity. A maximum for the erythema activity of sunlight is given as the relatively narrow range around 308 nm.

Numerous compounds are known for protecting against UV-B radiation; these are mostly derivatives of 3-benzylidenecamphor, of 4-aminobenzoic acid, of cinnamic acid, of salicylic acid, of benzophenone, and also of 2-phenylbenzimidazole.

It has long been incorrectly assumed that the long-wave UV-A radiation with a wavelength between 320 nm and 400 nm has only a negligible biological effect and that, correspondingly, the UV-B rays are responsible for most photodamage to the human skin. However, it has meanwhile been demonstrated by numerous studies that UV-A radiation is far more hazardous than UV-B radiation with regard to the triggering of photodynamic, specifically phototoxic, reactions and chronic changes in the skin. The harmful effect of the UV-B radiation can also be further intensified by UV-A radiation.

Thus, it has been proven, inter alia, that even UV-A radiation under entirely normal everyday conditions is sufficient to damage, within a short time, the collagen and elastin fibers which are of essential importance for the structure and firmness of the skin. This results in chronic light-induced skin changes—the skin “ages” prematurely. The clinical appearance of skin aged by light includes, for example, wrinkles and lines and an irregular, furrowed relief. In addition, the areas affected by light-induced skin aging may have irregular pigmentation. The formation of brown spots, keratoses and even carcinomas or malignant melanomas is also possible. Skin aged prematurely by everyday exposure to UV is additionally characterized by a lower activity of the Langerhans cells and slight chronic inflammation.

In addition, even very low radiation doses can trigger photochemical reactions. These include, in particular, the formation of free radicals, which in turn can trigger uncontrolled secondary reactions as a result of their high reactivity. In order to prevent such reactions, antioxidants and/or free-radical scavengers can additionally be added to the cosmetic or dermatological formulations. Thus, for example, it has been proposed to use vitamin E, a substance with known antioxidative effect in light protection formulations, although the effect achieved here falls a long way short of the desired effect.

Approximately 90% of the ultraviolet radiation which reaches the earth consists of UV-A rays. Whereas UV-B radiation varies greatly depending on a large number of factors (for example season and time of day or latitude), UV-A radiation remains relatively constant from day to day irrespective of seasonal and diurnal or geographic factors. At the same time, most of the UV-A radiation penetrates into the living epidermis, while approximately 70% of UV-B rays are retained by the horny layer.

Sunbathing is regarded by most people as pleasurable, and the disadvantageous consequences are initially not taken into consideration. However, in recent years, knowledge about the negative effects of excessively intensive solar irradiation has emerged, for which reason a greater number and more strongly protecting sunscreens are used.

Since the contributions of the various wavelength ranges of UV light to light-induced skin changes have not been fully explained, it is nowadays increasingly assumed that preventive protection against both UV-A and also UV-B rays, for example by applying light protection filter substances in the form of a cosmetic or dermatological formulation to the skin, is of fundamental importance. Cosmetic or dermatological compositions are intended, when applied to the skin in a thin layer, to protect it against the negative effects of solar radiation.

Emulsions too play a decisive role in the field of sunscreen formulations. In this connection, preference is given to flowable emulsions since they can be applied more easily than viscous creams. Most sunscreens are applied in the vicinity of water or during sporting activity (perspiration), for which reason particular importance is to be attributed to the water resistance of such formulations. A water-resistant sunscreen product protects the user not only after bathing, but also protects him against sunburn during bathing.

In order to achieve high sunscreen factors coupled with very good water resistance, W/O formulations are usually advantageous. However, W/O emulsions often have unsatisfactory cosmetic properties: during application, they may leave behind a greasy, shiny and sometimes sticky impression on the skin and—particularly on hairy skin—are difficult to spread.

By contrast, O/W emulsions have a less greasy effect on the skin, have instead a matting effect and absorb more rapidly into the skin. They are generally perceived by the consumer as easier and more cosmetically elegant than W/O emulsions. Since water is the external phase, O/W emulsions are, however, usually only water resistant to a limited extent.

As well as the effect of the base, the binding capacity of the UV filter in or on the skin is also of great importance for the water resistance of the formulation. It will be appreciated that oil-soluble UV filters are bonded better to the (lipophilic) surface of the skin, or can be washed off therefrom with more difficulty than water-soluble UV filters.

An object of the invention was therefore to obtain, in a simple and cost-effective manner, preparations (in particular O/W formulations) which are characterized by good water resistance. In particular, the aim was to find preparations which have a high content of water-soluble UV filters and nevertheless have very good water resistance.

The use of film-forming, water-soluble or water-dispersible polymers in cosmetic or dermatological formulations is known per se. Thus, for example, the company brochure TDS-248 from B.F. Goodrich Company describes the use of various types of polymers based on polyacrylate or on polyurethane in cosmetic formulations, for example for improving the feel on the skin. However, this publication was unable to point the way to the present invention.

It was surprising and could not have been foreseen by the person skilled in the art that the use of film-forming, water-soluble or water-dispersible polyurethanes for improving the water resistance of cosmetic or dermatological formulations comprising at least one customary UV filter substance would overcome the disadvantages of the prior art.

In addition, it was surprising that the use of film-forming, water-soluble or water-dispersible polyurethanes for improving the water resistance of O/W formulations would overcome the disadvantages of the prior art.

It was also surprising that the use of film-forming, water-soluble or water-dispersible polyurethanes for improving the water resistance of cosmetic or dermatological formulations comprising at least one customary water-soluble UV filter substance would overcome the disadvantages of the prior art.

Polyurethanes are polymers in the macromolecules of which the repeat units are linked by urethane groups —NH—CO—O—. Polyurethanes are generally obtained by polyaddition from di—or polyhydric alcohols and isocyanates:

n HO—R¹—OH+n O═C═N—R²—N═C═O

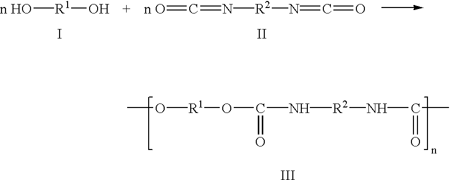

here, R¹ and R² may be low molecular weight or themselves even polymeric aliphatic or aromatic groups.

For the purposes of the present invention, advantageous polyurethanes are water-soluble or water-dispersible anionic polyurethanes (polyurethanes A) of

a) at least one compound which contains two or more active hydrogen atoms per molecule,

b) at least one diol containing acid or salt groups and

c) at least one diisocyanate,

which have a glass transition temperature of at least 15° C. and acid values in the range from 12 to 150, preferably 30 to 90, and the salts thereof.

Component a) is, in particular, a diol, aminoalcohol, diamine, polyesterol, polyetherol having a number-average molecular weight of in each case up to 3 000 or mixtures thereof, where up to 3 mol % of said compounds can be replaced by triols or triamines. Preference is given to diols and polyesterdiols. In particular, component (a) comprises at least 50% by weight, based on the total weight of component (a), of a polyesterdiol. Suitable polyesterdiols are all those which are customarily used for the preparation of polyurethanes, in particular reaction products of phthalic acid and diethylene glycol, isophthalic acid and 1,4-butanediol, isophthalic acid/adipic acid and 1,6-hexanediol, and adipic acid and ethylene glycol or 5-NaSO₃-isophthalic acid, phthalic acid, adipic acid and 1,6-hexanediol.

Examples of diols preferred according to the invention are ethylene glycol, propylene glycol, butylene glycol, neopentyl glycol, polyetherols, such as, for example, polyethylene glycols having molecular weights up to 3 000, block copolymers of ethylene oxide and propylene oxide having number-average molecular weights of up to 3 000 or block copolymers of ethylene oxide, propylene oxide and butylene oxide which contain the copolymerized alkylene oxide units in random distribution or in the form of blocks. Particular preference is given to ethylene glycol, neopentyl glycol, di-, tri-, tetra-, penta- and/or hexaethylene glycol. Poly(α-hydroxycarboxylic acid)diols are also advantageous for the purposes of the present invention.

Advantageous aminoalcohols are, for example, 2-aminoethanol, 2-(N-methylamino)-ethanol, 3-aminopropanol or 4-aminobutanol.

Advantageous diamines are, for example, ethylenediamine, propylenediamine, 1,4-diaminobutane and 1,6-diaminohexane, and α,ω-diamines which can be prepared by amination of polyalkylene oxides with ammonia.

Component b) is, in particular, a dimethylolpropanoic acid or compounds of the formulae

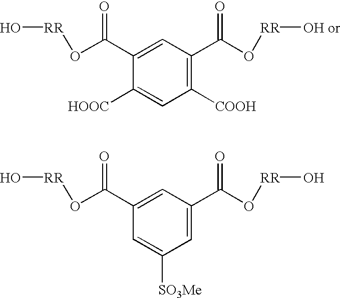

in which RR is in each case a C₂-C₁₈-alkylene group, and Me is Na or K.

Component c) is, in particular, hexamethylene diisocyanate, isophorone diisocyanate, methyl diphenylisocyanate (MDI) and/or tolylene diisocyanate.

The polyurethanes are obtainable by reacting the compounds of groups a) and b) with the compounds of group c) under an inert atmosphere in an inert solvent at temperatures of from 70 to 130° C. This reaction can, where appropriate, be carried out in the presence of chain extenders in order to prepare polyurethanes with relatively high molecular weights. As is customary in the preparation of polyurethanes, the components [(a)+(b)]:(c) are advantageously used in the molar ratio from 0.8 to 1.1:1. The acid number of the polyurethane is determined by the composition and the concentration of the compounds of component (b) in the mixture of components (a) and (b).

The polyurethanes have K values in accordance with H. Fikentscher (determined in 0.1% strength by weight solutions in N-methylpyrrolidone at 25° C. and pH=7) of from 15 to 100, preferably 25 to 50.

The K value, also referred to as intrinsic viscosity, is a parameter for characterizing polymers which can be easily determined via viscosity measurements of polymer solutions and is therefore frequently used in the industrial sector. For a certain type of polymer, it is assumed to be dependent only on the average molar mass of the sample under investigation under standardized measuring conditions and is calculated from the equation K value=1000 k according to the Fikentscher equation

$$k = \frac{1.5 l g {η}_{r} - 1 \pm \sqrt{1 + ( \frac{2}{c} + 2 + 1.5 l g {η}_{r} ) \cdot 1.5 l g {η}_{r}}}{150 + 300 c}$$

in which:

ηᵣ=relative viscosity (dynamic viscosity of the solution/dynamic viscosity of the solvent) and c=mass per unit volume of polymer in the solution (in g/cm³).

Following neutralization, the polyurethanes containing acid groups are (partially or completely) soluble in water or are dispersible without the assistance of emulsifiers. Generally, the salts of the polyurethanes have better solubility in water or dispersibility in water than the nonneutralized polyurethanes. Bases which can be used for the neutralization of the polyurethanes are alkali metal bases, such as sodium hydroxide solution, potassium hydroxide solution, sodium carbonate, sodium hydrogencarbonate, potassium carbonate or potassium hydrogencarbonate, and alkaline earth metal bases, such as calcium hydroxide, calcium oxide, magnesium hydroxide or magnesium carbonate, and ammonia and amines. Polyurethanes containing acid groups have proven particularly useful for the neutralization of 2-amino-2-methylpropanol, diethylaminopropylamine and triisopropanolamine. The neutralization of the polyurethanes containing acid groups can also be carried out using mixtures of two or more bases, e.g. mixtures of sodium hydroxide solution and triisopropanolamine. Depending on the intended use, the neutralization can be partial, e.g. to an extent of 20 to 40%, or complete, i.e. to an extent of 100%.

These polymers and their preparation are described in more detail in DE-A-42 25 045, to the entire scope of which reference is made here.

For the purposes of the present invention, also advantageous are water-soluble or water-dispersible, cationic polyurethanes and polyureas of

a) at least one diisocyanate, which may already have been reacted beforehand with one or more compounds which contain two or more active hydrogen atoms per molecule, and

b) at least one diol, primary or secondary aminoalcohol, primary or secondary diamine or primary or secondary triamine having one or more tertiary, quaternary or protonated tertiary amino nitrogen atoms,

which have a glass transition temperature of at least 25° C. and an amine number of from 50 to 200, based on the nonquaternized or protonated compounds, and the salts thereof. The amine number is preferably in the range from 65 to 180, in particular 70 to 170, particularly preferably 75 to 160 and very particularly 80 to 150.

Preferred diisocyanates are those given for the polyurethanes A. Compounds having two or more active hydrogen atoms are diols, aminoalcohols, diamines, polyesterols, polyamidodiamines and polyetherols. Advantageous compounds of this type are those given for the polyurethanes A.

The polyurethanes are prepared as described for the polyurethanes A. Charged cationic groups can be generated in the polyureas from the present tertiary amino nitrogen atoms either by protonation, e.g. with carboxylic acids such as lactic acid, or by quaternization, e.g. using alkylating agents such as C₁- to C₄-alkyl halides or -sulfates. Examples of such alkylating agents are ethyl chloride, ethyl bromide, methyl chloride, methyl bromide, dimethyl sulfate and diethyl sulfate.

These polymers and their preparation are described in more detail in DE-A-42 41 118, to the entire contents of which reference is hereby made.

Also advantageous for the purposes of the present invention are linear polyurethanes with carboxylate groups of

a) a 2,2-hydroxymethyl-substituted carboxylic acid of the formula

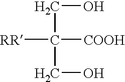

in which RR′ is a hydrogen atom or a C₁-C₂₀-alkyl group, which is used in an amount which is sufficient for 0.35 to 2.25 milliequivalents of carboxyl groups per g of polyurethane to be present in the polyurethane,

b) 10 to 90% by weight, based on the weight of the polyurethane, of one or more organic compounds having no more than two active hydrogen atoms and

c) one or more organic diisocyanates.

The carboxyl groups present in the polyurethane are, finally, at least partially neutralized with a suitable base. These polymers and their preparation are described in EP-A-619 111, to the entire contents of which reference is hereby made.

Also advantageous for the purposes of the present invention are carboxyl-containing polycondensation products with glass transition temperatures of >20° C. from anhydrides of tri- or tetracarboxylic acids and diols, diamines or aminoalcohols (polyesters, polyamides or polyester amides). These polymers and their preparation are described in more detail in DE-A-42 24 761, to the entire contents of which reference is hereby made.

The polymers used according to the invention preferably have a K value of from 25 to 100, preferably 25 to 50.

For the purposes of the invention, advantageous examples are polyurethane-1 and/or polyurethane-4.

Particularly advantageous polyurethanes for the purposes of the present invention are the grades obtainable under the trade name Avalure™ UR from B. F. Goodrich company, such as, for example, Avalure™ UR 445, Avalure™ UR 450 and the like. Also advantageous for the purposes of the present invention is also the polyurethane obtainable under the trade name Luviset Pur from BASF.

Cosmetic or dermatological preparations for the purposes of the present invention comprise—based on the total weight of the preparations—advantageously 0.1 to 5% by weight, in particular 0.2 to 2.5% by weight, of film-forming polyurethanes.

The cosmetic and/or dermatological formulations according to the invention can have the customary composition and be used for cosmetic and/or dermatological light protection, and also for the treatment, care and cleansing of the skin and/or of the hair and as a make-up product in decorative cosmetics. Correspondingly, the preparations according to the invention can, depending on their composition, be used, for example, as skin protection cream, cleansing milk, sunscreen lotion, nourishing cream, day cream or night cream etc. In some instances, it is possible and advantageous to use the preparations according to the invention as a base for pharmaceutical formulations. Preference is given, in particular, to those cosmetic and dermatological preparations which are in the form of a skincare or make-up product.

For years, the cosmetic and dermatological preparations according to the invention are applied to the skin and/or the hair in a sufficient amount in the manner customary for cosmetics.

For the purposes of the present invention, it is also advantageous to create cosmetic and dermatological preparations whose main purpose is not protection against sunlight, but which nevertheless has a content of UV protection substances. Thus, for example, UV-A and/or UV-B filter substances are often incorporated, for example, into day creams or make-up products. UV protection substances, like antioxidants and, if desired, preservatives, also represent effective protection of the preparations themselves against deterioration. Also favorable are cosmetic or dermatological preparations in the form of a sunscreen composition.

Accordingly, the preparations for the purposes of the present invention preferably comprise, in addition to one or more UV filter substances according to the invention, additionally at least one further UV-A and/or UV-B filter substance. The formulations may, although it is not obligatory, also optionally comprise one or more organic and/or inorganic pigments as UV filter substances which may be present in the water phase and/or the oil phase.

Preferred inorganic pigments are metal oxides and/or other metal compounds which are insoluble or virtually insoluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides.

For the purposes of the present invention, such pigments may advantageously be treated on the surface (“coated”), the intention being, for example, to form or retain an amphiphilic or hydrophobic character. This surface treatment can consist in providing the pigments with a thin hydrophobic layer by methods known per se.

According to the invention, titanium dioxide pigments coated with octylsilanol are, for example, advantageous. Suitable titanium dioxide particles are obtainable under the trade name T805 from Degussa. Also particularly advantageous are TiO₂ pigments coated with aluminum stearate, e.g. both available under the trade name MT 100 T from TAYCA.

A further advantageous coating of the inorganic pigments consists of dimethylpolysiloxane (also: dimethicone), a mixture of completely methylated, linear siloxane polymers which are terminally blocked with trimethylsiloxy units. Particularly advantageous for the purposes of the present invention are zinc oxide pigments coated in this way.

Also advantageous is a coating of the inorganic pigments with a mixture of dimethylpolysiloxane, in particular dimethylpolysiloxane with an average chain length of from 200 to 350 dimethylsiloxane units, and silica gel, which is also referred to as simethicone. It is particularly advantageous if the inorganic pigments are additionally coated with aluminum hydroxide or aluminum oxide hydrate (also: alumina, CAS No.: 1333-84-2). Titanium dioxides which have been coated with simethicone and alumina are particularly advantageous, it being possible for the coating to also contain water. One example thereof is the titanium dioxide obtainable under the trade name Eusolex T2000 from Merck.

An advantageous organic pigment for the purposes of the present invention is 2,2′-methylenebis(6-(2H-benzotriazol-2-yl)-4-(1,1,3,3-tetramethylbutyl)phenol) [INCI: Bis-octyltriazole], which is characterized by the chemical structural formula

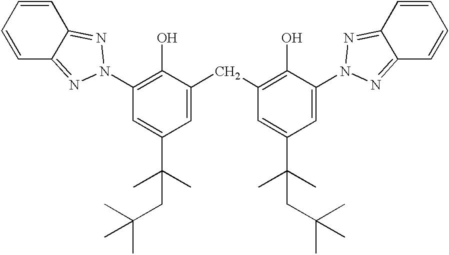

and is available under the trade name Tinosorb® M from CIBA-Chemikalien GmbH.

Preparations according to the invention advantageously comprise substances which absorb UV radiation in the UV-A and/or UV-B region, where the total amount of filter substances is, for example, 0.1% by weight to 30% by weight, preferably 0.5 to 20% by weight, in particular 1.0 to 15.0% by weight, based on the total weight of the preparations, in order to make available cosmetic preparations which protect the hair or the skin from the entire range of ultraviolet radiation. They can also be used as sunscreen compositions for the hair or the skin.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Further advantageous UV-A filter substances are phenylene-1,4-bis(2-benzimidazyl)-3,3′-5,5′-tetrasulfonic acid

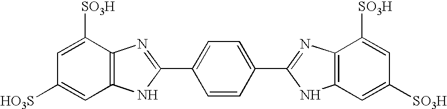

and its salts, particularly the corresponding sodium, potassium or triethanolammonium salts, in particular the phenylene-1,4-bis(2-benzimidazyl)-3,3′-5,5′-tetrasulfonic acid bis-sodium salt

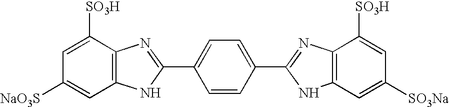

having the INCI name Bisimidazylate, which is obtainable, for example, under the trade name Neo Heliopan AP from Haarmann & Reimer.

Also advantageous are 1,4-di(2-oxo-10-sulfo-3-bornylidenemethyl)benzene and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid) and is characterized by the following structure:

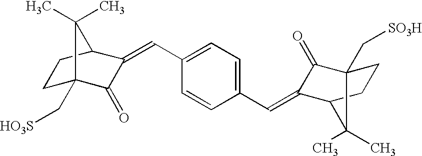

Advantageous UV filter substances for the purposes of the present invention are also broadband filters, i.e. filter substances which absorb both UV-A and also UV-B radiation.

Advantageous broadband filters or UV-B filter substances are, for example, bis-resorcinyltriazine derivatives having the following structure:

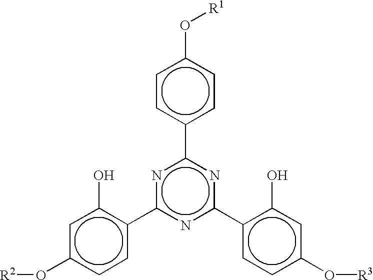

where R¹, R² and R³, independently of one another, are chosen from the group of branched and unbranched alkyl groups having 1 to 10 carbon atoms or represent a single hydrogen atom. Particular preference is given to 2,4-bis{[4-(2-ethylhexyloxy)-2-hydroxy]phenyl}-6-(4-methoxyphenyl)-1,3,5-triazine (INCI: Aniso Triazine), which is obtainable under the trade name Tinosorb® S from CIBA-Chemikalien GmbH.

Particularly advantageous preparations for the purposes of the present invention, which are characterized by a high or very high UV-A protection, preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′-5,5′-tetrasulfonic acid and/or its salts, 1,4-di(2-oxo-10-sulfo-3-bornylidenemethyl)benzene and/or salts thereof and/or 2,4-bis{[4-(2-ethyl-hexyloxy)-2-hydroxy]phenyl}-6-(4-methoxyphenyl)-1,3,5-triazine, in each case individually or in any combinations with one another.

Other UV filter substances which have the structural formula

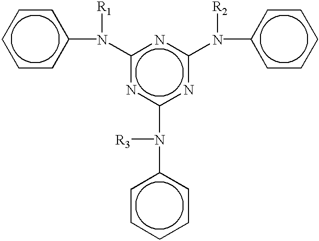

are also advantageous UV filter substances for the purposes of the present invention, for example the s-triazine derivatives described in European laid-open specification EP 570 838 A1, the chemical structure of which is given by the generic formula

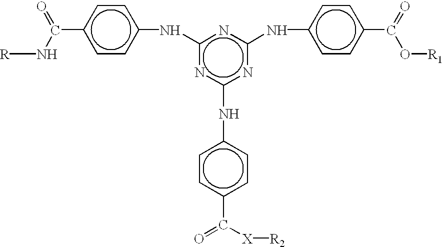

where

R is a branched or unbranched C₁-C₁₈-alkyl radical, a C₅-C₁₂-cycloalkyl radical, optionally substituted by one or more C₁-C₄-alkyl groups,

X is an oxygen atom or an N H group,

R¹ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

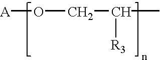

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

R₂ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

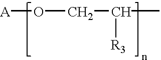

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

if X is an oxygen atom.

A particularly preferred UV filter substance for the purposes of the present invention is also an asymmetrically substituted s-triazine, the chemical structure of which is given by the formula

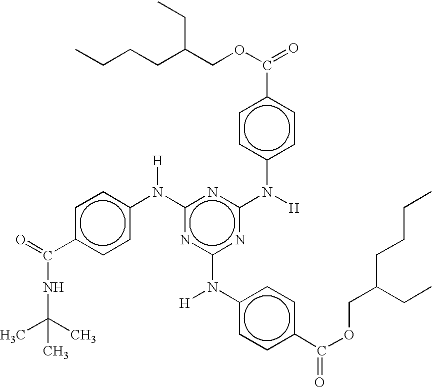

which is also referred to below as dioctylbutylamidotriazone (INCI: Dioctylbutamidotriazone) and is obtainable under the trade name UVASORB HEB from Sigma 3V.

Also advantageous for the purposes of the present invention is a symmetrically substituted s-triazine, tris(2-ethylhexyl) 4,4′,4″-(1,3,5-triazine-2,4,6-triyltriimino)tris-benzoate, synonym: 2,4,6-tris[anilino(p-carbo-2′-ethyl-1′-hexyloxy)]-1,3,5-triazine (INCI: Octyl Triazone), which is sold by BASF Aktiengesellschaft under the trade name UVINUL® T 150.

European laid-open specification 775 698 also describes preferred bis-resorcinyltriazine derivatives, the chemical structure of which is given by the generic formula

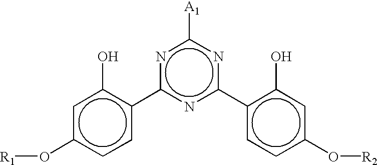

where R₁, R₂ and A₁ represent very different organic radicals.

Also advantageous for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethyl-carboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethyl-hexyloxy)-2-hydroxy]phenyl}-6-(1-methylpyrrol-2-yl)-1,3,5-triazine, 2,4-bis{[4-tris(trimethyl-siloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxy-phenyl)-1,3,5-triazine.

An advantageous broadband filter for the purposes of the present invention is 2,2′-methylenebis(6-(2H-benzotriazol-2-yl)-4-(1,1,3,3-tetramethylbutyl)phenol), which is characterized by the chemical structural formula

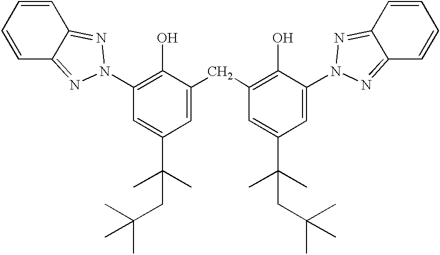

and is obtainable under the trade name Tinosorb® M from CIBA-Chemikalien GmbH.

An advantageous broadband filter for the purposes of the present invention is also 2-(2H-benzotriazol-2-yl)-4-methyl-6-[2-methyl-3-[1,3,3,3-tetramethyl-1-[(trimethylsilyl)-oxy]disiloxanyl]propyl]phenol (CAS No.: 155633-54-8) with the INCI name Drometrizole Trisiloxane, which is characterized by the chemical structural formula.

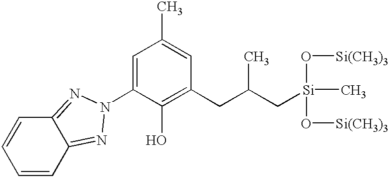

The UV-B and/or broadband filters may be oil-soluble or water-soluble. Advantageous oil-soluble UV-B and/or broadband filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxybenzo-phenone

and UV filters bonded to polymers.

Advantageous water-soluble UV-B and/or broadband filter substances are, for example:

salts of 2-phenylbenzimidazole-5-sulfonic acid, such as its sodium, potassium or its triethanolammonium salt, and the sulfonic acid itself;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)-sulfonic acid and salts thereof.

A further light protection filter substance which can be used advantageously according to the invention is ethylhexyl 2-cyano-3,3-diphenylacrylate (octocrylene), which is obtainable from BASF under the name Uvinul® N 539 and is characterized by the following structure:

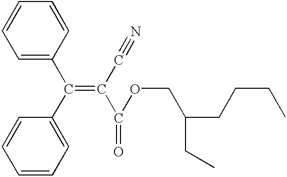

It may also be considerably advantageous to use polymer-bound or polymeric UV filter substances in preparations according to the present invention, in particular those described in WO-A-92/20690.

In addition, in some instances it may be advantageous to incorporate further UV-A and/or UV-B filters into cosmetic or dermatological preparations according to the invention, for example certain salicylic acid derivatives, such as 4-isopropylbenzyl salicylate, 2-ethylhexyl salicylate (=octyl salicylate), homomenthyl salicylate.

The list of UV filters mentioned which can be used for the purposes of the present invention is not of course intended to be limiting.

The preparations according to the invention advantageously comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair or the skin from the entire range of ultraviolet radiation. They can also be used as sunscreens for the hair or the skin.

The cosmetic and dermatological preparations for the purposes of the present invention are preferably dispersed two- or multi-phase systems which, in addition to one or more oil phases, may additionally comprise one or more water phases. They may be in the form of cosmetic or dermatological emulsions—for example of the W/O, O/W, W/O/W, O/W/O type and the like. Such emulsions may preferably also be a microemulsion, a Pickering emulsion or a sprayable emulsion. Particular preference for the purposes of the present invention is, however, given to formulations in which water is the external phase, in particular O/W emulsions, hydrodispersions or aqueous systems which comprise water-soluble UV filter substances.

The cosmetic and dermatological preparations for the purposes of the present invention may comprise cosmetic auxiliaries as are customarily used in such preparations, e.g. preservatives, bactericides, perfumes, antifoams, dyes, pigments which have a coloring action, thickeners, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

The amounts of cosmetic or dermatological auxiliaries and carrier substances and perfume to be used in each case can be easily determined by simple exploratory experiments by the person skilled in the art depending on the type of product in question.

An additional content of antioxidants is generally preferred. Favorable antioxidants which may be used according to the invention are all antioxidants customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, gutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoxime compounds (e.g buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyro-phenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives threof (e.g. stilbene oxide, trans-stilbene oxide) and the derivatives (salts, esters, ethers, sugars, nucleotides, nucleosides, peptides and lipids) of said active ingredients which are suitable according to the invention.

The amount of the abovementioned antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant(s), it is advantageous to choose the respective concentrations thereof from the range 0.001 to 10% by weight, based on the total weight of the formulation.

If vitamin A, or vitamin A derivatives, or carotenes or derivatives thereof are the antioxidant(s), it is advantageous to choose the respective concentrations from the range 0.001 to 10% by weight, based on the total weight of the formulation.

The oil phase of the preparations for the purposes of the present invention is advantageously chosen from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, from the group of esters of aromatic carboxylic acids and saturated and/or unsaturated, branched and/or unbranched alcohols of chain length from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, be advantageously chosen from the group of synthetic, semisynthetic and natural oils, e.g. olive oil, sunflower oil, soybean oil, peanut oil, rapeseed oil, almond oil, palm oil, coconut oil, palm kernel oil and the like.

For the purposes of the present invention, any mixtures of such oil and wax components can also be used advantageously. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, capryl/caproic triglyceride, dicaprylyl ether and dicaprylyl carbonates.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase may also advantageously have a content of cyclic or linear silicone oils or consist entirely of such oils, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils can also be used advantageously for the purposes of the present invention, for example hexamethylcyclotrisiloxane, polydimethylsiloxane, poly(methylphenylsiloxane).

Other particularly advantageous mixtures are those of cyclomethicone and isotridecyl isononanoate, and of cyclomethicone and 2-ethylhexyl isostearate.

The aqueous phase of the preparations according to the invention optionally advantageously comprises

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol and, in particular one or more thickeners which can be advantageously chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of Carbopols, for example Carbopol grades 980, 981, 1 382, 2 984, 5 984, in each case individually or in combination.

The examples below serve to illustrate the present invention without limiting it. Unless stated otherwise, all amounts, proportions and percentages given are based on the weight and the total amount or on the total weight of the preparations.

### EXAMPLES

1. PIT—Sun Sprays

| | 1 | 2 | 3 | 4 | 5 |
|---|---|---|---|---|---|
| Glycerol Monostearate SE | 0.50 | | 3.00 | 2.00 | 4.00 |
| Ceteareth-12 | | 5.00 | | 1.00 | 1.50 |
| Ceteareth-20 | | | | 2.00 | |
| Ceteareth-30 | 5.00 | | 1.00 | | |
| Stearyl Alcohol | | | 3.00 | | 0.50 |
| Cetyl Alcohol | 2.50 | 1.00 | | 1.50 | |
| Ethylhexyl Methoxycinnamate | | | | 5.00 | 8.00 |
| Aniso Triazine | | 1.50 | | 2.00 | 2.50 |
| Butyl Methoxydibenzoylmethane | | | 2.00 | | |
| Dioctyl Butamidotriazone | 1.00 | | | 2.00 | 2.00 |
| Ethylhexyl Triazone | 4.00 | | 3.00 | 4.00 | |
| 4-Methylbenzylidene Camphor | | 4.00 | | | 2.00 |
| Octocrylene | | 4.00 | | | 2.50 |
| Bisimidazylate | | | 0.50 | | 1.50 |
| Phenylbenzmidazole Sulfonic Acid | 0.50 | | | 3.00 | |
| Bisoctyltriazole | | 2.50 | | 1.00 | |
| C12-15 Alkyl Benzoate | | 2.50 | | | 5.00 |
| Dicaprylyl Ether | | | 3.50 | | |
| Butylene Glycol Dicaprylate/ | 5.00 | | | 6.00 | |
| Dicaprate | | | | | |
| Dicaprylyl Carbonate | | | 6.00 | | 2.00 |
| Dimethicone | | 0.50 | 1.00 | | |
| Phenyltrimethicone | 2.00 | | | 0.50 | 0.50 |
| Shea Butter | | 2.00 | | | 0.50 |
| PVP Hexadecene Copolymer | 0.50 | | | 0.50 | 1.00 |
| Glycerol | 3.00 | 7.50 | 5.00 | 7.50 | 2.50 |
| Vitamin E Acetate | 0.50 | | 0.25 | | 1.00 |
| Polyurethane | 0.20 | 0.50 | 1.50 | 0.50 | 0.40 |
| DMDM Hydantoin | 0.60 | | 0.40 | 0.20 | |
| Konkaben LMB ® | | 0.20 | | | 0.15 |
| Methylparaben | | 0.50 | 0.25 | 0.15 | |
| Phenoxyethanol | 0.50 | 0.40 | | 1.00 | 0.60 |
| Ethanol | 3.00 | 2.00 | 1.50 | | 1.00 |
| Perfume | q.s. | q.s. | q.s. | q.s. | q.s. |
| Water | ad. 100 | ad. 100 | ad. 100 | ad. 100 | ad. 100 |

2. O/W Sunscreen Emulsions

| | 1 | 2 | 3 | 4 | 5 | 6 | 7 |
|---|---|---|---|---|---|---|---|
| Glycerol Monostearate SE | 0.50 | 1.00 | 3.00 | | | 1.50 | |
| Glycerol Stearate Citrate | 2.00 | | | 1.00 | 2.00 | | 2.50 |
| Stearic acid | | 3.00 | | 2.00 | | | |
| PEG-40 Stearate | 0.50 | | | | | 2.00 | |
| Cetyl Phosphate | | | | | 1.00 | | |
| Stearyl Alcohol | | | 3.00 | | | 2.00 | 0.50 |
| Cetyl Alcohol | 2.50 | 1.00 | | 1.50 | 0.50 | | 2.00 |
| Ethylhexyl Methoxycinnamate | | | | 5.00 | 6.00 | | 8.00 |
| Aniso Triazine | | 1.50 | | 2.00 | 2.50 | | 2.50 |
| Butyl Methoxydibenzoyl- | 1.00 | | 2.00 | | | 2.00 | |
| methane | | | | | | | |
| Dioctyl Butamidotriazone | | 1.00 | | 3.00 | | 2.00 | |
| Ethylhexyl Triazone | 4.00 | | 3.00 | 4.00 | 4.00 | 2.00 | |
| 4-Methylbenzylidene Camphor | 4.00 | 4.00 | | | 2.00 | 4.00 | 2.00 |
| Octocrylene | | 4.00 | | | | | 2.50 |
| Bisoctyltriazole | 1.00 | | | 2.00 | 1.00 | | |
| Bisimidazylate | 1.00 | | 0.50 | | | 1.00 | 1.50 |
| Phenylbenzmidazole Sulfonic | 0.50 | | | 3.00 | | | |
| Acid | | | | | | | |
| Titanium Dioxide | 1.00 | 1.50 | | 3.00 | 2.00 | 2.00 | |
| C12-15 Alkyl Benzoate | | 2.50 | | | 4.00 | 7.00 | 5.00 |
| Dicaprylyl Ether | | | 3.50 | | 2.00 | | |
| Butylene Glycol | 5.00 | | | 6.00 | | | |
| Dicaprylate/Dicaprate | | | | | | | |
| Dicaprylyl Carbonate | | | 6.00 | | | 2.00 | 2.00 |
| Dimethicone | | 0.50 | 1.00 | | 2.00 | | |
| Cyclomethicone | 2.00 | | | 0.50 | | | 0.50 |
| Shea Butter | | 2.00 | | | | | 0.50 |
| PVP Hexadecene Copolymer | 0.50 | | | 0.50 | 1.00 | | 1.00 |
| Glycerol | 3.00 | 7.50 | | 7.50 | 5.00 | | 2.50 |
| Xanthan gum | 0.15 | | 0.05 | | | | 0.30 |
| Sodium Carbomer | | 0.20 | 0.10 | 0.20 | | | |
| Vitamin E Acetate | 0.50 | | 0.25 | | 0.75 | | 1.00 |
| Polyurethane | 0.50 | 0.20 | 1.50 | 0.50 | 0.60 | 1.00 | 0.40 |
| DMDM Hydantoin | | 0.60 | 0.40 | 0.20 | | | |
| Konkaben LMB ® | | | | 0.18 | 0.20 | 0.10 | 0.15 |
| Methylparaben | 0.15 | | 0.25 | | 0.50 | | |
| Phenoxyethanol | 1.00 | 0.40 | | 0.40 | 0.50 | 0.40 | 0.60 |
| Ethanol | | 2.00 | 1.50 | | 3.00 | | 1.00 |
| Perfume | q.s. | q.s. | q.s. | q.s. | q.s. | q.s. | q.s. |
| Water | ad. | ad. | ad. | ad. | ad. | ad. | ad. |
| | 100 | 100 | 100 | 100 | 100 | 100 | 100 |

3. Hydrodispersions

| | 1 | 2 | 3 | 4 | 5 |
|---|---|---|---|---|---|
| Ceteareth-20 | 1.00 | | | 0.5 | |
| Cetyl Alcohol | | | 1.00 | | |
| Sodium Carbomer | | 0.20 | | 0.30 | |
| Acrylates/C10-30 | 0.50 | | 0.40 | 0.10 | 0.10 |
| Alkyl Acrylate | | | | | |
| Crosspolymer | | | | | |
| Xanthan Gum | | 0.30 | 0.15 | | 0.50 |
| Ethylhexyl Methoxycinnamate | | | | 5.00 | 8.00 |
| Aniso Triazine | | 1.50 | | 2.00 | 2.50 |
| Butyl Methoxydibenzoylmethane | 1.00 | | 2.00 | | |
| Dioctyl Butamidotriazone | | 2.00 | | 2.00 | 1.00 |
| Ethylhexyl Triazone | 4.00 | | 3.00 | 4.00 | |
| 4-Methylbenzylidene Camphor | 4.00 | 4.00 | | | 2.00 |
| Octocrylene | | 4.00 | 4.00 | | 2.50 |
| Bisoctyltriazole | 1.00 | | | 2.00 | |
| Bisimidazylate | 1.00 | | 0.50 | | 2.00 |
| Phenylbenzmidazole Sulfonic | 0.50 | | | 3.00 | |
| Acid | | | | | |
| Titanium dioxide | 0.50 | | 2.00 | 3.00 | 1.00 |
| C18-36 Acid Triglyceride | | 2.00 | | 1.00 | |
| C12-15 Alkyl Benzoate | 2.00 | 2.50 | | | |
| Dicaprylyl Ether | | 4.00 | | | |
| Butylene Glycol | 4.00 | | 2.00 | 6.00 | |
| Dicaprylate/Dicaprate | | | | | |
| Dicaprylyl Carbonate | | 2.00 | 6.00 | | |
| Dimethicone | | 0.50 | 1.00 | | |
| Phenyltrimethicone | 2.00 | | | 0.50 | 2.00 |
| Shea Butter | | 2.00 | | | |
| PVP Hexadecene Copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine Soja | | | 1.50 | | |
| Vitamin E Acetate | 0.50 | | 0.25 | | 1.00 |
| Polyurethane | 0.15 | 0.60 | 1.50 | 1.00 | 0.80 |
| DMDM Hydantoin | | 0.60 | 0.40 | 0.20 | |
| Konkaben LMB ® | 0.20 | | | | 0.15 |
| Methylparaben | 0.50 | | 0.25 | 0.15 | |
| Phenoxyethanol | 0.50 | 0.40 | | 1.00 | 0.60 |
| Ethanol | 3.00 | 2.00 | 1.50 | | 1.00 |
| Perfume | q.s. | q.s. | q.s. | q.s. | q.s. |
| Water | ad. 100 | ad. 100 | ad. 100 | ad. 100 | ad. 100 |

4. W/O Sunscreen Emulsions

| | 1 | 2 | 3 | 4 | 5 |
|---|---|---|---|---|---|
| Cetyldimethicone Copolyol | | 2.50 | | 4.00 | |
| Polyglyceryl-2 | 5.00 | | | | 4.50 |
| dipolyhydroxystearate | | | | | |
| PEG-30 dipolyhydroxystearate | | | 5.00 | | |
| Ethylhexyl Methoxycinnamate | | 8.00 | | 5.00 | 4.00 |
| Aniso Triazine | 2.00 | 2.50 | | 2.00 | 2.50 |
| Butyl Methoxydibenzoylmethane | | | 2.00 | 1.00 | |
| Dioctyl Butamidotriazone | 3.00 | 1.00 | | | 3.00 |
| Ethylhexyl Triazone | | | 3.00 | 4.00 | |
| 4-Methylbenzylidene Camphor | | 2.00 | | 4.00 | 2.00 |
| Octocrylene | 7.00 | 2.50 | 4.00 | | 2.50 |
| Bisoctyltriazole | 1.00 | | | 2.00 | |
| Bisimidazylate | 1.00 | 2.00 | 0.50 | | |
| Phenylbenzmidazole Sulfonic | 0.50 | | | 3.00 | 2.00 |
| Acid | | | | | |
| Titanium dioxide | | 2.00 | 1.50 | | 3.00 |
| Mineral oil | | | 10.0 | | 8.00 |
| C12-15 Alkyl Benzoate | | | | 9.00 | |
| Dicaprylyl Ether | 10.00 | | | | 7.00 |
| Butylene Glycol | | | 2.00 | 8.00 | 4.00 |
| Dicaprylate/Dicaprate | | | | | |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Shea Butter | | | 3.00 | | |
| PVP Hexadecene Copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine Soja | | 1.00 | 1.50 | | |
| MgSO₄ | 1.00 | 0.50 | | 0.50 | |
| MgCl₂ | | | 1.00 | | 0.70 |
| Vitamin E Acetate | 0.50 | | 0.25 | | 1.00 |
| Polyurethane | 0.10 | 0.60 | 1.50 | 1.00 | 0.80 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | 0.60 |
| Ethanol | 3.00 | | 1.50 | | 1.00 |
| Perfume | q.s. | q.s. | q.s. | q.s. | q.s. |
| Water | ad. 100 | ad. 100 | ad. 100 | ad. 100 | ad. 100 |

1. Method of using film-forming, water-soluble or water-dispersible polyurethanes for improving the water resistance of cosmetic or dermatological formulations comprising at least one customary UV filter substance.

2. Method of using film-forming, water-soluble or water-dispersible polyurethanes for improving the water resistance of O/W formulations.

3. The method as claimed in claim 2, wherein the O/W formulation is an emulsion.

4. The method as claimed in claim 2, wherein the O/W formulation is a microemulsion.

5. The method as claimed in claim 2, wherein the O/W formulation is a hydrodispersion.

6. Method of using film-forming, water-soluble or water-dispersible polyurethanes for improving the water resistance of cosmetic or dermatological formulations comprising at least one customary water-soluble UV filter substance.

7. The method as claimed in claim 1, wherein the cosmetic or dermatological preparations comprise, based on their total weight, from 0.1 to 10% by weight of polyurethanes.

8. The method as claimed in claim 1, wherein the cosmetic or dermatological light protection preparations comprise, as UV-A filter substance, one or more water-soluble UV-A filter substances, particularly water-soluble UV-A filter substances chosen from the group consisting of phenylene-1,4-bis(2-benzimidazyl)-3,3′-5,5′-tetrasulfonic acid and/or 1,4-di(2-oxo-10-sulfo-3-bornylidenemethyl)benzene and/or salts thereof, particularly the corresponding sodium, potassium or triethanolammonium salts and/or the corresponding 10-sulfato compounds, in particular phenylene-1,4-bis(2-benzimidazyl)-3,3′-5,5′-tetrasulfonic acid bis-sodium salt.

9. The method as claimed in claim 1, wherein at least one broadband filter is chosen from the group of bis-resorcinyltriazine derivatives, in particular 2,4-bis{[4-(2-ethylhexyloxy)-2-hydroxy]phenyl}-6-(4-methoxyphenyl)-1,3,5-triazine.

10. The method as claimed in claim 1, wherein at least one broadband filter is chosen from the group of benzotriazole derivatives, in particular 2,2′-methylenebis(6-(2H-benzotriazol-2-yl)-4-(1,1,3,3-tetramethylbutyl)phenol) and/or 2-(2H-benzotriazol-2-yl)-4-methyl-6-[2-methyl-3-[1 ,3,3,3-tetramethyl-1-[(trimethylsilyl)-oxy]disiloxanyl]propyl]phenol.
